(19) United States
(12) Patent Application Publication
(10) Pub. No.: US 2002/0127598 A1
(43) Pub. Date: Sep. 12, 2002

(54) Solution-phase combinatorial library synthesis and pharmaceutically active compounds produced thereby

(76) Inventors: Wenqiang Zhou, Montreal (CA); Sathya Upendran, St-Laurent (CA); Radhakrishnan P. Iyer, Shrewsbury, MA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/820,087

(22) Filed: Mar. 27, 2001

Related U.S. Application Data
(60) Provisional application No. 60/192,193, filed on Mar. 27, 2000.

(51) Int. Cl. G01N 33/53; C07H 21/04; C07H 21/02; C07D 473/16; C07D 473/18

(52) U.S. Cl. 435/7.1; 536/23.1; 544/277; 544/269; 544/276; 544/319

(57) ABSTRACT

The invention provides methods for solution-phase synthesis of nucleotide-based compounds and new libraries of such compounds. Compounds of the invention are useful for a variety of therapeutic applications, including treatment of viral or bacterial infections and associated diseases and disorders.

### BACKGROUND OF THE INVENTION

1. Field of the Invention

The invention provides libraries of nucleotide-based compounds and solution-phase methods for synthesis of such libraries. Compounds of the invention are useful for a variety of therapeutic applications, including treatment of viral or bacterial infections and associated diseases and disorders.

2. Background

The important initial step in the development of therapeutic agents is the discovery of compounds that bind to a protein, enzyme or receptor of interest. Through careful structure/activity work of resulting active compounds, one arrives at a lead compound for further development into a clinical candidate. This traditional process of drug discovery is a long and arduous endeavor. Often it takes 10 to 15 years before a new drug makes it into the marketplace.

Recent advances in molecular biology and genomics have led to identification of new molecular targets for drug discovery. As a result of the limitation of traditional drug discovery, new approaches to the discovery of therapeutics have been developed. In the more modem approaches, large libraries of diverse compounds are synthesized by a number of methods and subjected to high throughput in vitro screening against a particular molecular target implicated in a disease. The active compounds so identified are then subjected to Structure-Activity Relationship (SAR) work to eventually identify the lead compound.

Modem drug discovery approaches entail the synthesis and screening of libraries of compounds. The design and synthesis of such libraries is often based on a unique molecular skeleton or scaffold. By incorporating a variety of structural elements into a scaffold, local as well as global molecular diversity can be achieved which facilitates obtaining specific interactions between a ligand and its receptor. The structural elements contribute to molecular diversity by variable spatial display of ionic, hydrogen-bonding, charge-transfer and van der Waals interactions thus allowing for the selection of the best ‘fit’ between the ligand and its receptor.

Traditionally, libraries have been constructed using solid-support synthesis methods, such as synthesis of a library on ‘beads’. Solid support methods are useful because reactive products can be readily isolated in a relatively pure form by simply washing away excess reagents and solvents from the support matrix, something that is not possible with solution based methods.

However, solid phase synthesis procedures are limited in several ways. The yield of compounds produced by such a method are restricted by the amount of solid support required and the loading capacity of the solid support. Additionally solid support procedures require functionalized supports and substrates, compatible spacer or linker arms for attaching the reactive groups to the support, capping strategies for blocking unreacted products and do not permit purification of resin-bound intermediates.

Solution-phase synthesis strategies can overcome many of the limitations of a solid-phase approach and can provide highly pure materials (>90-95%) regardless of reaction efficiencies. The most common techniques for solution-phase synthesis involve liquid/liquid or solid/liquid extraction to purify the compounds after each reaction step. Compound libraries may be produced using a parallel synthesis method, wherein each compound is synthesized in a separate reaction vessel. Alternatively, a split-and-pool strategy may be used, wherein pools of compounds are prepared with each pool containing a different fixed building block at a particular location. The pool with the highest activity is then selected and a new batch of compound pools is synthesized with an additional fixed building block at another location. Iterative rounds of assay and synthesis are repeated until all of the positions are defined.

See also: An, H. et al., Solution Phase Combinatorial Chemistry. Synthesis of Novel Linear Pyridinopolyamine Libraries with Potent Antibacterial Activity. J. Org. Chem. 62:5156-5164 (1997); Barone, A. D. et al., In situ Activation of bis-dialkylaminopbosphines-a new method for synthesizing deoxyoligonucleotides on polymer supports. Nuc. Acids Res. 12(10): 4051-4061 (1984); Bleczinski, C. F. et al., Steroid-DNA Interactions Increasing Stability, Sequence-Selectivity, DNA/RNA Discrimination, and Hypochromicity of Oligonucleotide Duplexes. J. Am. Chem. Soc. 121:10889-10894 (1999); Boger, D. L. et al., Generalized Dipeptidomimetic Template: Solution Phase Parallel Synthesis of Combinatorial Libraries. J. Am. Chem. Soc. 118: 2109-2110 (1996); Carell, T. et al., A Novel Procedure for the Synthesis of Libraries Containing Small Organic Molecules. Angew. Chem. Int. Ed. Engl. 33(20): 2059-2061 (1994); Cheng, S. et al., Novel Solution Phase Strategy for the Synthesis of Chemical Libraries Containing Small Organic Molecules. J. Am. Chem. Soc. 118: 2567-2573 (1996); Gallop, M. A. et al., Applications of Combinatorial Technologies to Drug Discovery. 1. Background and Peptide Combinatorial Libraries. J. Med. Chem. 37(9): 1233-1251 (1994); Houghten, R. A. et al., Mixture-Based Synthetic Combinatorial Libraries. J. Med. Chem. 42(19): 3743-3778 (1999); Iyer, R. P. et al., 3H-1,2-Benzodithiole-3-one 1, I -Dioxide as an Improved Sulfurizing Reagent in the Solid-Phase Synthesis of Oligodeoxyribonucleoside Phosphorothioates. J. Am. Chem. Soc. 112: 1253-1254 (1990); Iyer, R. P. et al., Nucleoside Oxazaphospholidines as Novel Synthons in Oligonucleotide Synthesis. J. Org. Chem. 60: 5388-5389 (1995); Jyer, R. P. et al., Solid-Phase Stereoselective Synthesis of Oligonucleotide Phosphorothioates: The Nucleoside Bicyclic Oxazaphospholidines as Novel Synthons. Tetrahedron Lefts. 39: 2491-2494 (1998); Iyer, R. P. and Beaucage, S. L. In Comprehensive Natural Products Chemistry Vol. 7: DNA and Aspects of Molecular Biology, Knol, E. T. , Ed. Oligonucleotide Synthesis. Elsevier Science. London 1999, pages 105-152; Thompson, L. A. et al., Synthesis and Applications of Small Molecule Libraries. Chem. Rev. 96: 555-600 (1996); Zhou, W. et al., Combinatorial Synthesis Using Nucleic Acid-Based (NABTM) Scaffold: Parallel Solid-Phase Synthesis of Nucleotide Libraries. Tetrahedron Letts. 0: 1-5 (1999); and Gordon, E. M. et al., Applications of Combinatorial Technologies to Drug Discovery. 2. Combinatorial Organic Synthesis, Library Screening Strategies, and Future Directions. J. Med. Chem. 37(10): 1385-1401 (1994).

### SUMMARY OF THE INVENTION

We have now found new nucleotide-based compounds that are useful for a variety of therapeutic applications, including to treat against viral or bacterial infections.

The invention also provides new methods for synthesis of nucleotide-based compounds and new libraries of such compounds. In particular, the invention provides new methods for construction of compound libraries utilizing a nucleic acid-based (NAB) scaffold. This approach enables incorporating structural elements that can provide both “sequence-specific” interactions (e.g., hydrogen-bonding interactions between nucleobases) as well as “shape-specific” motifs (e.g., bulges and stem-loop structures) that can allow specific recognition of other nucleic acids and proteins. Libraries based on NAB scaffold can potentially mimic the molecular recognition that exists between cellular macromolecules The invention provides methods for constructing compound libraries by a solution-phase synthesis approach.

Other aspects of the invention are disclosed infra.

### BRIEF DESCRIPTION OF THE DRAWINGS

FIG. 1 shows the results of herpes simplex virus (HSV-1) plaque reduction assay (PRA) for the specified compounds. The results in FIG. 1 correspond to the data given in Table 3.

FIG. 2 shows the results of herpes simplex virus (HSV-1) plaque reduction assay (PRA) for the specified compounds. The results in FIG. 2 correspond to the data given in Table 4.

FIG. 3 shows the results of herpes simplex virus (HSV-1) plaque reduction assay (PRA) for the specified compounds. The results in FIG. 3 correspond to the data given in Table 5.

FIG. 4 shows the results of herpes simplex virus (HSV-1) plaque reduction assay (PRA) for the specified compounds. The results in FIG. 4 correspond to the data given in Table 6.

FIG. 5 shows the results of herpes simplex virus (HSV-1) plaque reduction assay (PRA) for the specified compounds. The results in FIG. 5 correspond to the data given in Table 7.

### DETAILED DESCRITPTION OF THE INVENTION

As discussed above, we have discovered new methods for construction of a compound library. Preferred library members include compounds of the following Formula I or I′:

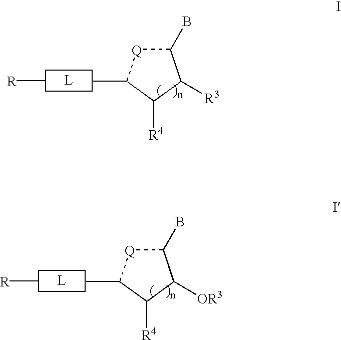

wherein L is a linking group such as e.g. an amide, ester, diester or the like, or an optionally substituted alkylene (e.g. C₁₋₂₀ alkylene), optionally substituted alkenylene (e.g., C₂₋₂₀ alkenylene) or alkynylene (e.g., C₂₋₂₀ alkynylene) having such groups either as a chain member of pendant to the chain, and which may be optionally substituted with one or more substituents selected from a group consisting of O, S, Se, NR¹NR², CR¹CR², OR, SR and SeR (R, R¹ and R² defined below), or an enzymatically reactive (particularly, cleavable) moiety such as an amide, ester, and the like;

Q is carbon or a heteroatom such as O, S or N;

R is hydrogen or a hydroxyl group or a hydrophobic group, e.g. a moiety having from 1 to about 18 carbon atoms, such as optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;

R¹, R², R³ and R⁴ are each independently selected from a group as defined by R;

B is optionally substituted adenine, optionally substituted thymidine, optionally substituted cytosine or an optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or B is heteroaromatic or heteroalicyclic group other than an adenine, thymidine, cytosine or guanine and preferably has from 1 to 3 separate or fused rings and 1 to 3 N, O or S atoms;

n is an integer of from 1 to 5 and where n is greater than 1 designates that corresponding additional carbon ring or acyclic members are present (i.e. where n is 2 an additional carbon ring member (to form a 6-membered ring) or acyclic carbon is present; where n is 3, two additional carbon ring members (to form a 7-membered ring) or acyclic carbon is present, and so on);

and pharmaceutically acceptable salts thereof.

In the above Formulae I and I′, it is understood that the dashed line indicates the ring may be in the open or closed configuration.

The depicted sugar group may be natural or modified (e.g. synthetic) form, or in an open chain form (where one of the depicted ring bonds would not be present).

Preferred R groups of compounds of formulae I and I′ include cyclic groups, particularly alicyclic groups that may comprise one or more single or polycyclic rings, particularly a bridged or fused ring structure, with 0, 1 or 2 endocyclic carbon-carbon double bonds. Additional preferred R groups include heteroalicyclic moieties, particularly heteroalicyclic groups having from 5 to about 8 ring member, preferably with one or two O, N or S ring members, particularly one or two oxygen ring members.

Preferred compounds of the invention include those of formulae I and I′ where the nucleoside is linked to the R group via a phosphorous group at the 5′ end. Such linkages could also be established via the 2′ or 3′ sites of the nucleoside. When R is a nucleoside, linkages can be via 5′ to 3′, 5′ to 5′, 3′ to 3′, 2′ to 5′ and 2′ to 2′, or any combination thereof, of the participating nucleosides.

Preferred compounds of the invention include those of the following Formulae II and II′, having the depicted configurations:

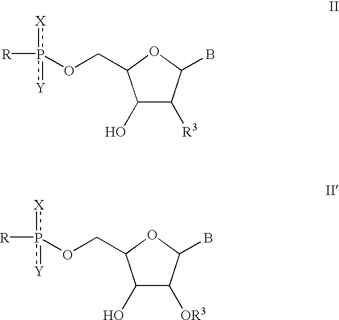

wherein X and Y are each independently selected from a group consisting of O, S, Se, NR¹NR², CR¹CR², OR, SR and SeR, or one or both of X and Y are an enzymatically reactive (particularly, cleavable) moiety such as an amide, ester, and the like;

R is hydrogen or a hydrophobic group, e.g. a moiety having from 1 to about 18 carbon atoms, such as optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;

R¹, R² and R³ are each independently selected from a group as defined by R;

B is optionally substituted adenine, optionally substituted thymidine, optionally substituted cytosine or an optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or a heterocyclic structure that is covalently linked to the sugar ring;

and pharmaceutically acceptable salts thereof.

In the above Formulae II and II′, it is understood that the dashed line extending to each of the substituents X and Y designates that one, but not both, of X and Y may have an additional chemical bond (i.e. a double bond).

The depicted sugar group may be natural or modified (e.g. synthetic) form, or in an open chain form (where one of the depicted ring bonds would not be present).

Preferred R groups of compounds of formulae II and II′ include cyclic groups, particularly alicyclic groups that may comprise one or more single or polycyclic rings, particularly a bridged or fused ring structure, with 0, 1 or 2 endocyclic carbon-carbon double bonds. Additional preferred R groups include heteroalicyclic moieties, particularly heteroalicyclic groups having from 5 to about 8 ring member, preferably with one or two O, N or S ring members, particularly one or two oxygen ring members.

As mentioned above, either one or both of X and Y may be an enzymatically reactive group, i.e. the group may be cleavable or otherwise reactive in vivo upon administration to a mammal, particularly a human. Preferred enzymatically reactive groups include e.g. amides (which may be cleaved in vivo with an amidase), esters (which may be cleaved in vivo with an esterase), and acetal and ketal groups.

Preferred compounds of the invention include those of formulae II and II′ where the nucleoside is linked to the R group via a phosphorous group at the 5′ end. Such linkages could also be established via the 2′ or 3′ sites of the nucleoside. When R is a nucleoside, linkages can be via 5′ to 3′, 5′ to 5′, 3′ to 3′, 2′ to 5′ and 2′ to 2′, or any combination thereof, of the participating nucleosides.

Preferably, compounds of the invention will be present in enantiomerically enriched mixtures, i.e. where one enantiomer is present in a greater amount than other stereoisomer(s) of the compound, particularly where one enantiomer is present in amount of at least about 60 mole percent, relative to all stereoisomers present of the compound; preferably where one enantiomer is present in amount of at least about 70 or 80 mole percent, relative to all stereoisomers present of the compound; still more preferably where one enantiomer is present in amount of at least about 85, 90, 92, 95, 96, 97, 98 or 99 mole percent, relative to all stereoisomers present of the compound.

Particularly preferred compounds of the invention include those of the following Formulae III and III′, having the depicted configurations:

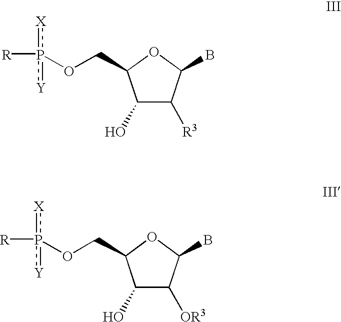

wherein X, Y, R, R¹, R², R³, B and n are the same as defined above for Formulas II and II′; and pharmaceutically acceptable salts thereof.

In the above Formulae III and III′, it is understood that the dashed line extending to each of the substituents X and Y designates that one, but not both, of X and Y may have an additional chemical bond (i.e. a double bond).

In the above Formulae III and III′, the depicted sugar group may be natural or modified (e.g. synthetic) form, or in an open chain form (where one of the depicted ring bonds would not be present).

In the above Formulae I, I′, II, II′, III and III′, alkyl groups preferably contain from 1 to about 18 carbon atoms, more preferably from 1 to about 12 carbon atoms and most preferably from 1 to about 6 carbon atoms. Specific examples of alkyl groups include, for example, methyl, ethyl, n-propyl, isopropyl, n-butyl, t-butyl, isobutyl, pentyl, hexyl, heptyl, octyl, nonyl, decyl, etc.

In the above Formulae I, I′, II, II′, III and III′, aralkyl groups include the above-listed alkyl groups substituted by a carbocyclic aryl group having 6 or more carbons, for example, phenyl, naphthyl, phenanthryl, anthracyl, etc.

In the above Formulae I, I′, II, II′, III and III′, cycloalkyl groups preferably have from 3 to about 8 ring carbon atoms, e.g. cyclopropyl, cyclopentyl, cyclohexyl, cycloheptyl, 1,4-methylenecyclohexane, adamantyl, cyclopentylmethyl, cyclohexylmethyl, 1- or 2-cyclohexylethyl and 1-, 2- or 3-cyclohexylpropyl, etc.

In the above Formulae I, I′, II, II′, III and III′, exemplary heteroaromatic and heteroalicyclic group include pyridyl, pyrazinyl, pyrimidyl, furyl, pyrrolyl, thienyl, thiazolyl, oxazolyl, imidazolyl, indolyl, benzothiazolyl, tetrahydrofuranyl, tetrahydropyranyl, piperidinyl, morpholino and pyrrolidinyl.

Mononucleotides of compounds of the invention (compounds of Formulae I, I′, II, II′, III and III′) invention include adenine, cytodine, guanosine and thymidine.

Polynucleotides of compounds of the invention (compounds of Formulae I, I′, II, II′, III and III′) preferably contain from about 1 to about 20 mononuculeotides, more preferably from 1 to about 10 mononuculeotides and still more preferably from 1 to about 5 mononuculeotides. The polynucleotides are suitably constructed such that the 5′ group of one mononucleotide pentose ring is attached to the 3′ group of its neighbor in one direction via, for example, a phosphodiester or a phosphorthioate internucleotide linkage.

Sugar groups of compounds of the invention may be comprised of mono-, di-, oligo- or poly-saccharides wherein each monosaccharide unit comprises from 3 to about 8 carbons, preferably from 3 to about 6 carbons, containing polyhydroxy groups or polyhydroxy and amino groups. Non-limiting examples include glycerol, ribose, fructose, glucose, glucosamine, mannose, galactose, maltose, cellobiose, sucrose, starch, amylose, amylopectin, glycogen and cellulose. The hydroxyl and amino groups are present as free or protected groups containing e.g. hydrogens and/or halogens. Preferred protecting groups include acetonide, t-butoxy carbonyl groups, etc. Monosaccharide sugar groups may be of the L or D configuration and a cyclic monosaccharide unit may contain a 5 or 6 membered ring of the a Or D conformation. Disaccharides may be comprised of two identical or two dissimilar monosaccharide units. Oligosaccharides may be comprised of from 2 to 10 monosaccharides and may be homopolymers, heteropolymers or cyclic polysugars. Polysaccharides may be homoglycans or heteroglycans and may be branched or unbranched polymeric chains. The di-, oligo- and poly-saccharides may be comprised of 1→4, 1→6 or a mixture of 1→4 and 1→6 linkages. The sugar moiety may be attached to the link group through any of the hydroxyl or amino groups of the carbohydrate.

Preferred compounds of the invention comprise R groups containing one of the primary or secondary alcohol structures represented in Table 1 below.

Preferred library syntheses of the invention are carried out using a solution-phase synthesis approach. The solution-phase approach allowed the preparation of larger quantities of materials and the production of a wider variety of compounds than was achievable by solid-phase synthesis methods. The reaction conditions for parallel solution-phase synthesis were devised so as to optimize the coupling reactions for quantitative yield and to obtain purified materials from the reaction mixture.

The solution-phase synthesis approach was used to construct a representative 150-member library. Nucleoside building blocks were derived from deoxy- and ribonucleosides 2a-f and the R groups were derived from 25 primary and secondary alcohols each with differing degrees of hydrophobicity, as well as steric and electronic properties (Table 1). The R groups were coupled to the nucleoside units via phosphorothioate linkages so as to increase the stability of the compounds against nuclease-mediated degradation, provide ionic properties to the compounds and to increase the aqueous solubility of the compounds.

Compounds of the invention can be prepared as generally depicted in Scheme 1. In addition to the synthetic route depicted in Scheme 1, library compounds may also be assembled using a phosphodiester approach, a phosphortriester approach and/or the H-phosphonate methodology (R. P. Jyer et al. In Comprehensive Natural Products, D. H. R. Barton and K. Nakanishi Eds., Elsevier Science Vol 7, in press). In Scheme 1 below, preferred stereoisomers and substituent groups are depicted, although it is understood that other compounds of the invention can be produced by the same or similar procedures.

SCHEME 1

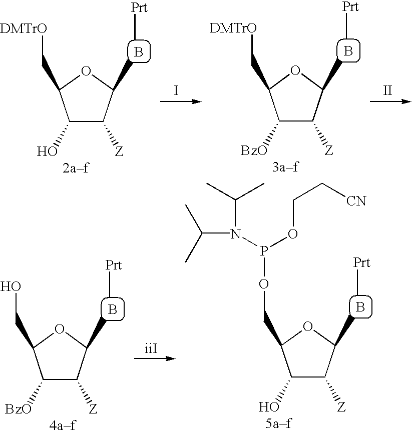

| | |
|---|---|
| a: Z = Hydrogen; B = adenine b: Z = Hydrogen; B = cytosine c: Z = Hydrogen; B = guanine | 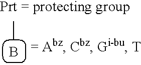 |
| d: Z = Hydrogen; B = thymine | A = adenine |
| e: Z = OMe; B = cytosine | C = cytosine |
| f: Z = OMe; B = guanine | G = guanine |
| i = BzCl/pyridene | T = thymine |
| ii = trifloroacetic acid/CHCl₃ | Bz = benzoyl |
| iii = bis-(N,N-diisopropylamino)-2-cyanoethylphosphine, | i-bu = isobutyryl |
| diisopropylammonium tetrazolide in CH₂Cl₂ | DMTr = 4,4′-dimethoxytriphenylmethyl |

Scheme 1 shows the preparation of nucleoside building blocks 5a-f from 2a-f via 3,4a-f. Nucleosides 2a-f are commercially available and were used for the preparation of the 3′-benzoate derivatives 3a-f. Unmasking of the 5′-DMTr group in 3a-f was achieved with a short exposure to trifluoroacetic acid to produce quantitative yields of 4a-f. Nucleosides 4a-f were then converted into the corresponding phosphoramidite derivatives 5a-f (Barome, A. D. et al., In situ activation of bis-dialkylaminophosphines—a new method for synthesizing deoxyoligonucleotides on polymer supports. Nuc. Acids Res. 12(10): 4051-4061 (1984); Iyer, R. P. et al., In Comprehensive Natural Products Chemistry Vol. 7:

DNA and Aspects of Molecular Biology, Knol, E. T., Ed. Oligonucleotide Synthesis. Elsevier Science. London 1999; pp 105-152). The phosphoramidites 5a-fwere characterized by NMR (¹H and ³¹P) and HRMS.

SCHEME 2

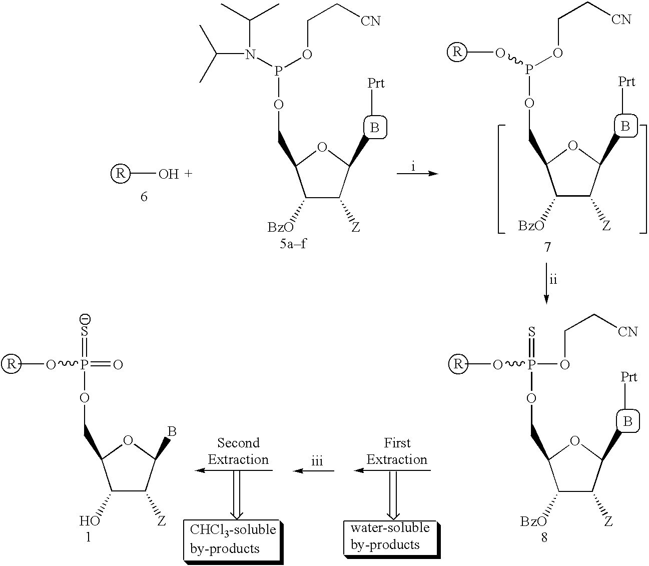

| | |
|---|---|
| a: Z = Hydrogen; B = adenine b: Z = Hydrogen; B = cytosine c: Z = Hydrogen; B = guanine | 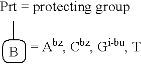 |
| d: Z = Hydrogen; B = thymine | A = adenine |
| e: Z = OMe; B = cytosine | C = cytosine |
| f: Z = OMe; B = guanine | G = guanine |
| i = 1H-Tetrazole in CH₃CN | T = Thymine |
| ii = 3H-1,2-benzodithiole-3-one-1,1-dioxide in CH₃CN | Bz = benzoyl |
| iii = 28% NH₄OH | i-bu = isobutyryl |
| \mathring{R} = structures are depicted in Table 1 | DMTr = 4,4′-dimethoxytriphenylmethyl |

Scheme 2 shows the preparation of a model library using nucleosides 5a-f and a set of six alcohols from table 1 (a 36 member library). The reactions were done on a 20-30 micromol scale in a parallel synthesis mode. The key coupling reaction was initiated by the addition of lH-tetrazole to a mixture consisting of each of the phosphoramidites 5a-f and the alcohol (Table 1). The resulting P(III) intermediates 7 were oxidatively sulfurized in situ by the addition of 3H-1,2-benzodithiole-3-one 1,1-dioxide (3H-BD) solution. The purification of the organic soluble triester intermediates 8 was achieved by partitioning between ethyl acetate and 5% sodium bicarbonate. It was anticipated that this process would facilitate the retention of the byproducts (diusopropyl ammonium tetrazolides and 1-benzothiole-2-oxa-3-one sulfoxides) in the aqueous phase. The Rₚ and Sₚ diastereomeric triesters 8 were then converted to product diesters 1 by treatment with 28% NH₄OH. Following this step, the purification of the crude product was achieved by simple extraction of the aqueous layer with chloroform which gave product diesters of greater than 85% purity as determined by analytical HPLC. A final desalting step using C-18 cartridges resulted in product diesters of greater than 90% purity as determined by analytical HPLC. Yields of the library members ranged from 80 to 85% starting from the amidites 5a-f. Analysis by NMR and MS confirmed the structures of the products. Mass spectral analysis further revealed that no base-modified products were obtained in the samples tested.

Conditions from the 36-member model library were used to produce a 150-member library using 25 alcohols (Table 1) and six nucleoside building blocks 5a-f (library 1). The quality of the resulting library was evaluated by analytical HPLC and each member was found to have purity greater than 90% following a final desalting step. These results are reported below in Table 2.

TABLE 1
Representative R groups used in construction of library
compounds. Twenty-five (i-xxv) primary and secondary alcohols were used to
construct 36- and 150-member representative libraries.

| Open-chain | Cyclic primary | Cyclic secondary | Aromatic |
|---|---|---|---|
| alchohol | alcohol | alcohol | alcohol |
| 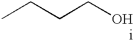 | 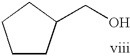 | 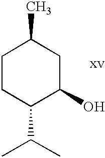 | 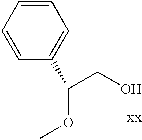 |
| 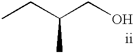 | 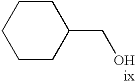 | 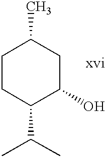 | 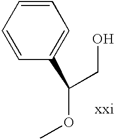 |
| 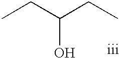 | 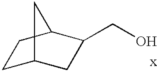 | 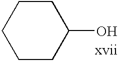 | 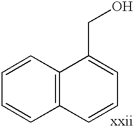 |
| 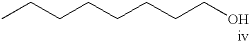 | 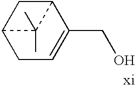 | 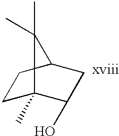 | 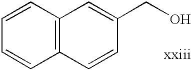 |
| 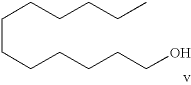 | 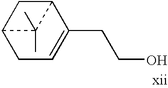 | 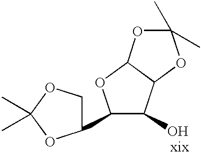 | 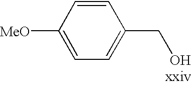 |
| 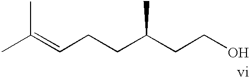 | 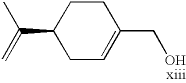 | | 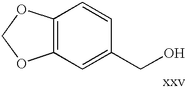 |
| 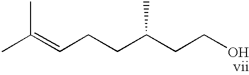 | 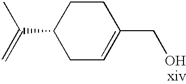 | | |

SCHEME 3

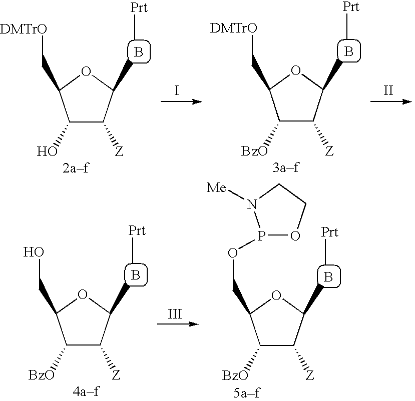

| | |
|---|---|
| a: Z = Hydrogen; B = adenine b: Z = Hydrogen; B = cytosine c: Z = Hydrogen; B = guanine | 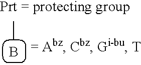 |
| d: Z = Hydrogen; B = thymine | A = adenine |
| e: Z = OMe; B = cytosine | C = cytosine |
| f: Z = OMe; B = guanine | G = guanine |
| i = BzCl/pyridene | T = thymine |
| ii = trifloroacetic acid/CHCl₃ | Bz = benzoyl |
| iii = 1,3-oxazaphospholidine chlorophosphite | i-bu = isobutyryl |
| | DMTr = 4,4′-dimethoxytriphenylmethyl |

SCHEME 4

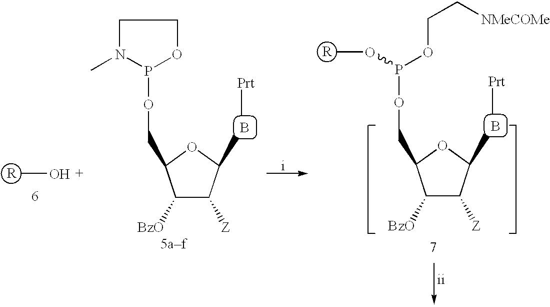

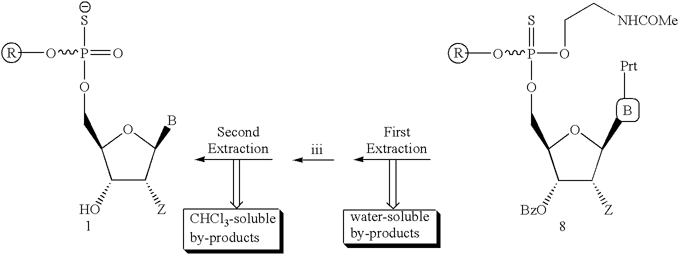

| | |
|---|---|
| a: Z = Hydrogen; B = adenine b: Z = Hydrogen; B = cytosine c: Z = Hydrogen; B = guanine | 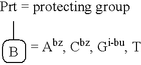 |
| d: Z = Hydrogen; B = thymine | A = adenine |
| e: Z = OMe; B = cytosine | C = cytosine |
| f: Z = OMe; B = guanine | G = guanine |
| i = 1H-Tetrazole in CH₃CN, followed by acetic anhydride | T = thymine |
| ii = 3H-1,2-benzodithiole-3-one-1,1-dioxide in CH₃CN | Bz = benzoyl |
| iii = 28% NH₄OH | i-bu = isobutyry1 |
| \mathring{R} = structures are depicted in Table 1 | DMTr = 4,4′-dimethoxytriphenylmethyl |

SCHEME 5

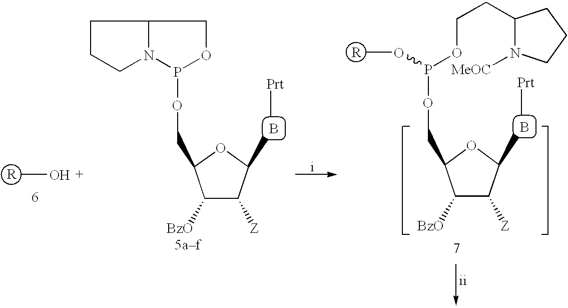

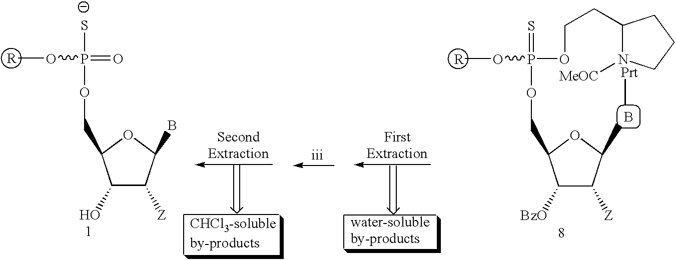

| | |
|---|---|
| a: Z = Hydrogen; B = adenine b: Z = Hydrogen; B = cytosine c: Z = Hydrogen; B = guanine | 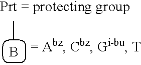 |
| d: Z = = Hydrogen; B = thymine | A = adenine |
| e: Z = OMe; B = cytosine | C = cytosine |
| f: Z = OMe; B = guanine | G = guanine |
| i = 1H-Tetrazole in CH₃CN, followed by acetic anhydride | T = thymine |
| ii = 3H-1,2-benzodithiole-3-one-1,1-dioxide in CH₃CN | Bz = benzoyl |
| iii = 28% NH₄OH | i-bu = isobutyryl |
| \mathring{R} = structures are depicted in Table 1 | DMTr = 4,4′-dimethoxytriphenylmethyl |

The method of library assembly described herein is adaptable to the solution-phase split and pool strategy for combinatorial synthesis. Other nucleoside building blocks such as nucleoside oxazaphospholidines can also be used in the preparation of library compounds to provide members with defined Rₚ and Sₚ stereochemistries.

Compound libraries of the invention preferably will contain at least about 2, 3, 4 or 5 distinct compounds, more preferably at least about 10 distinct compounds, still more preferably at least about 20, 30, 40, 50 ,60, 70, 80, 90 or 100 compounds, and may contain 200, 300, 400 or 500 or more compounds.

Compounds of the invention (compounds of Formulae I, I′, II, II′, III and III′) will be useful for a variety or therapeutic application, including in methods of treatment against infections and diseases associated with bacteria, fungi and viruses, which methods in general comprise administration of a therapeutically effective amount of one or more compounds of Formulae I, I′, II, II′, III and III′ to an infected animal, such a mammal, particularly a human.

Specifically, the invention includes methods of treatment of a mammal susceptible to (prophylactic treatment) or suffering from a disease associated with viruses, including retroviruses, DNA viruses and RNA viruses. More specifically, the invention includes methods of treatment of a mammal susceptible to (prophylactic treatment) or suffering from a disease associated with herpes viruses, hepatitis viruses, influenza viruses, immunodeficiency causing viruses, respiratory syncitia viruses, papilloma viruses and rhino viruses.

Particularly preferred are methods of treatment of a mammal susceptible to (prophylactic treatment) or suffering from a disease associated with viruses of the herpes family, e.g. herpes simplex viruses (HSV) including herpes simplex 1 and 2 viruses (HSV 1, HSV 2), varicella zoster virus (VZV; shingles), human herpes virus 6, cytomegalovirus (CMV), Epstein-Barr virus (EBV), and other herpes virus infections such as feline herpes infections, and diseases associated with hepatitis viruses including hepatitis B viruses (HBBV) virus. Examples of clinical conditions which are caused by such viruses include herpetic keratitis, herpetic encephalitis, cold sores and genital infections (caused by herpes simplex), chicken pox and shingles (caused by varicella zoster) and CMV-pneumonia and retinitis, particularly in immunocompromised patients including renal and bone marrow transplant and patients with Acquired Immune Deficiency Syndrome (AIDS). Epstein-Barr virus can cause infectious mononucleosis, and is also suggested as the causative agent of nasopharyngeal, immunoblastic lymphoma and Burkitt's lymphoma.

Compounds of the invention may be used as inhibitors of viral kinases, viral polymerases, and as disrupters of helicase-primase complexes with nucleic acids during viral replication.

Compounds of the invention also will be useful for cancer therapy, particularly to treat solid tumors, such as may be present in the liver, lung, brain or other tissue.

Compounds of the invention also will be useful for treatment against bacterial infections, including both Gram positive and Gram negative bacteria, and mycobacteria.

Administration of compounds of the invention may be made by a variety of suitable routes including oral, topical (including transdermal, buccal or sublingal), nasal and parenteral (including intraperitoneal, subcutaneous, intravenous, intradermal or intramuscular injection) with oral or parenteral being generally preferred. It also will be appreciated that the preferred method of administration and dosage amount may vary with, for example, the condition and age of the recipient.

Compounds of the invention may be used in therapy in conjunction with other pharmaceutically active medicaments, such as another anti-viral agent, or an anti-cancer agent. Additionally, while one or more compounds of the invention may be administered alone, they also may be present as part of a pharmaceutical composition in mixture with conventional excipient, i.e., pharmaceutically acceptable organic or inorganic carrier substances suitable for parenteral, oral or other desired administration and which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parenteral application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

Therapeutic compounds of the invention also may be incorporated into liposomes. The incorporation can be carried out according to known liposome preparation procedures, e.g. sonication and extrusion. Suitable conventional methods of liposome preparation are also disclosed in e.g. A. D. Bangham et al., J. Mol. Biol., 23:238-252 (1965); F. Olson et al., Biochim. Biophys. Acta, 557:9-23 (1979); F. Szoka et al., Proc. Nat. Acad. Sci., 75:4194-4198 (1978); S. Kim et al., Biochim. Biophys. Acta, 728:339-348 (1983); and Mayer et al., Biochim. Biophys. Acta, 858:161-168 (1986).

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests.

All documents mentioned herein are incorporated herein by reference.

The present invention is further illustrated by the following examples. These examples are provided to aid in the understanding of the invention and are not to be construed as limitations thereof.

### EXAMPLE 1

### Synthesis of Nucleoside Building Blocks (Schemes 1 and 3).

The nucleoside building blocks 5a-f were synthesized from commercially available nucleosides 2a-f via 3,4a-f as shown in scheme 1 and 3.

Step 1. Conversion of 2a-f to 3a-f.

Each of nucleosides 2a-f (4 millimol) was co-evaporated twice with anhydrous pyridine (50 mL). The reaction was placed in an ice bath, and benzoyl chloride (5 millimol) was added dropwise to the solution with stirring. The ice-bath was removed, and stirring continued for about 3 hours. The reaction mixture was then poured slowly into a flask containing ice-cold saturated aqueous sodium carbonate (500 mL) with vigorous stirring. The resulting white precipitate was collected and washed exhaustively with water to remove all pyridine. The products, 3a-f, were dried overnight in vacuo.

Step 2. Conversion of 3a-f to 4a-f.

Each of the nucleosides 3a-f obtained as above was dissolved in chloroform (200 mL) and treated with trifluoroacetic acid (20 millimol) for 10 minutes. The reaction mixture was then slowly poured into ice-cold saturated aqueous sodium carbonate (200 mL). The separated chloroform layer was twice extracted with sodium carbonate (5%), once with brine, and then dried over anhydrous sodium sulfate. Evaporation of the chloroform layer gave a residue which was either crystallized from ethyl acetate (when the nucleobase is a pyrimidine) or in other cases, purified by flash chromatography (methylene chloride/methanol, 95/5) to give 4a-f. The yield was 85-90% starting from 2a-f.

Step 3. Conversion of 4a-f to 5a-f.

NN-diisopropylammonium tetrazolide (1.5 millimol) and the 3 ′-O-benzoyl nucleoside derivative (3 millimol) 4a-f were dissolved in dry dichloromethane (100 mL). β-cyanoethyl tetraisopropyl phosphoramidite (3.3 millimol) was then added and the reaction mixture was stirred under argon for about 3 hours. The reaction mixture was stripped off dichloromethane in vacuo and ethyl acetate (200 mL) was added to the residue. The ethyl acetate layer was twice extracted with aqueous sodium carbonate (2%, 1 00 mL) and once with brine (100 mL). The organic layer was dried over anhydrous sodium sulfate and evaporated to give the crude amidites 5a-f. The crude products were purified by flash chromatography (silica gel, EtOAc/hexane/Et₃N, 74/25/1) to give 5a-f as white foam (yields 87-90%). The phosphoramidites 5a-f were characterized by NMR (¹H and ³¹P) and HRMS.

Typical spectral data are as follows:

Nucleoside 5a. ¹H NMR (CDCl₃): δ8.83 (m, 1H), 8.54 (m, 1 H), 8.03-8.10 (m, 4H), 7.27-7.64 (m, 6H), 6.68-6.74 (m, 1H), 5.73-5.78 (m, 1H), 4.51 (m, 1H), 3.81-4.11 (m, 4H), 3.61-3.65 (m, 2H), 2.67-3.01 (m, 4H), 1.18-1.22 (m, 12H) ppm; ³¹P NMR (CDCl₃): δ152.65 ppm; FABMS: Calcd. for C₃₃H₃₉N₇O₆P, 660.26995 (M+1); found m/z, 660.26979.

Nucleoside 5b: ¹H NMR (CD₃COCD₃): δ8.39-8.41 (m, 1H), 8.10-8.17(m 4H), 7.65-7.70 (m, 2H), 7.54-7.58 (m, 4H), 6.36-6.44 (m, 1H), 5.62-5.69 (m, 1H), 4.56-4.59 (m, 1H), 3.66-4.15 (m, 6H), 2.81-2.89 (m, 2H), 2.42-2.51 (m, 1H), 2.05 (m, 1H), 1.18-1.22 (m, 12H) ppm; ³¹P NMR (CD₃COCD₃): δ152.46, 152.28 ppm; FABMS: Calcd. for C₃₂H₃₉N₅O₇P, 636.25871 (M+1); found m/z, 636.25854.

Nucleoside 5c: ¹H NMR (CDCl₃): δ8.02-8.08 (m, 3H), 7.44-7.75 (m, 3H), 6.28-6.31 (m, 1H), 5.69-5.70 (m, 1H), 4.40 (m, 1H), 4.09-4.22 (m, 2H), 3.45-4.02 (m, 6H), 2.63-2.99 (m, 5H), 1.22-1.29 (m, 12H), 1.11-1.17 (m, 6H) ppm; ³¹P NMR (CDCl₃): δ152.67, 152.49 ppm; FABMS: Calcd. for C₃₀H₄₁N₇O₇P, 642.28051 (M+1); found m/z, 642.28068.

Nucleoside 5d: ¹H NMR (CD₃COCD₃): δ7.53-8.10 (m, 6H), 6.41-6.51 (m, 1H), 5.61-5.66 (m, 1H), 3.50-4.40 (m, 5H), 2.80-2.89 (m, 2H), 2.44-2.64 (m, 2H), 1.91-1.92 (m, 3H), 1.22-1.28 (m, 12H) ppm; ³¹P NMR (CD₃COCD₃): δ152.40, 152.06 ppm; FABMS: Calcd. for C₂₆H₃₆N₄O₇P, 547.23216 (M+1); found m/z, 547.23238.

Nucleoside 5e: 1H NMR (CD₃COCD₃): δ8.44-8.49 (m, 1H), 8.08-8.18 (m, 4H), 7.52-7.70 (m, 6H), 6.16-6.26 (m, 1H), 5.45-5.56 (m, 1H), 4.60-4.63 (m, 1H), 3.49-4.32 (m, 9H), 2.78-2.90 (m, 4H), 1.18-1.30 (m, 12H)ppm; ³¹P NMR(CD₃COCD₃): δ152.76, 152.46 ppm; FABMS: Calcd. for C₃₃H₄₁N₅O₈P, 666 (M+1); found m/z, 666.

Nucleoside 5f: ¹H NMR (CD₃COCD₃): δ8.15-8.25 (m, 3H), 7.53-7.74 (m, 3H), 6.04-6.27 (m, 1H), 4.61-4.70 (m, 2H), 3.90-4.11 (m, 4H), 3.70-3.77 (m, 2H), 1.21-1.28 (m, 18H)ppm, ³¹P NMR (CDCl₃): 6152.63, 152.55 ppm; FABMS: Calcd. for C₃₁H₄₃N₇O₈P, 672 (M+1); found m/z, 672.

### EXAMPLE 2

### Synthesis of a 150 Member (Products 1a-i to 1a-xxv) (See Schemes 2, 4 and 5, Table 1).

Each alcohol shown in Table 2 (i-xxv) (30 micromol) and each of the nucleoside amidites 5a-f (20 micromol) were added sequentially to a series of conical microtubes (2 mL, Ultident Scientific) containing lH-tetrazole solution in acetonitrile (1 mL, 100 micromol). The mixture was shaken in a platform shaker at room temperature for five minutes. Then 3H-BD (40 micromol) was added as a solid and the contents shaken for another 5 minutes. The acetonitrile was evaporated in a Speed Vac. Ethyl acetate (0.8 mL) was then added, followed by aqueous sodium bicarbonate (0.4 mL). Following thorough mixing of the phases, the organic layer containing the intermediate thiophosphate triester 8 was separated and evaporated to dryness. Aqueous ammonium hydroxide (28%, 1 mL) was added to the residue in each microtube. The tightly capped tubes were heated at 55° C for 3 hours. The aqueous ammoniacal solution was concentrated to dryness in a Speed Vac. The contents were dissolved in water (0.8 mL) and twice extracted with chloroform (0.4 mL). The aqueous layer was evaporated to dryness in a speed vac to obtain the library 1. Each member was obtained as a white solid. Quantitation was achieved on the basis of A₂₆₀ units, and the yields of product 1 were found in the range from 80 to 85% starting from 5a-f.

### EXAMPLE 3

### HPLC Analysis of Library Members 1 (Table 2).

Reversed-phase analytical HPLC was performed on a Waters 600 system equipped with a photodiode-array UV detector 996, autosampler 717, and Millenium 2000 software, using a Radial-Pak liquid chromatography cartridge [8 mm I.D., 8NVC 18]. Mobile phase: Buffer A: 0.1 M NH₄OAc; Buffer B: 20% A/80% CH₃CN v/v. Gradient: 100% A, 0-3 minutes; 40% A, 40 minutes; 100% B, 49 minutes).

TABLE 2
HPLC and ³¹P NMR data of representative library members.
Nucleosides 5a-f were combined with alcohols i-xxv to form a
150 member compound library. The compounds analyzed are represented
by the nucleoside (number and letter) followed by the Roman number
of the alcohol which corresponds to the numbers in Table 1.

| Library | HPLC | |
|---|---|---|
| Members | R₁ (Min) | ³¹P (ppm) |
| 1a-i | 30.5 | 58.97, 58.64 |
| 1b-i | 28.6 | 58.96 |
| 1c-i | 28.1 | 58.91, 58.61 |
| 1d-i | 29.7 | 58.32, 58.24 |
| 1a-xix | 36.0 | 59.39, 59.33 |
| 1b-xix | 35.1 | 59.75, 59.24 |
| 1c-xix | 33.6 | 59.36, 59.21 |
| 1d-xix | 38.2 | 59.72, 59.25 |
| 1e-xix | 33.7 | 59.45, 59.18 |
| 1f-xix | 32.5 | 59.30, 59.06 |
| 1a-xx | 35.1 | 59.09, 58.73 |
| 1c-xx | 32.6 | 59.06, 58.64 |
| 1b-xii | 47.5 | 58.91, 58.67 |
| 1d-xii | 48.2 | 58.97, 58.52 |
| 1e-xii | 46.1 | 59.00, 58.68 |

### EXAMPLE 4

### Biological Testing

HCMV.

Selected compounds of the invention were tested against human cytomeglovirus (HCMV). Briefly, a 96 well cell-based assay was used with human foreskin infected with HCMV strain with an MOI of 0.05 plaque forming units per ml. Each well was treated once with a 25 micromolar dose of test compound. Five days following treatment with the test compound, total cellular DNA was harvested after cell lysis. Cell lysates were applied to a Nylon membrane on a dot blot apparatus, the blots hybridized with a probe specific for HCMV DNA, and the blots scanned and analyzed using Scan analysis software. Tested compounds showed significant inhibition of viral growth relative to control samples.

HSV-1.

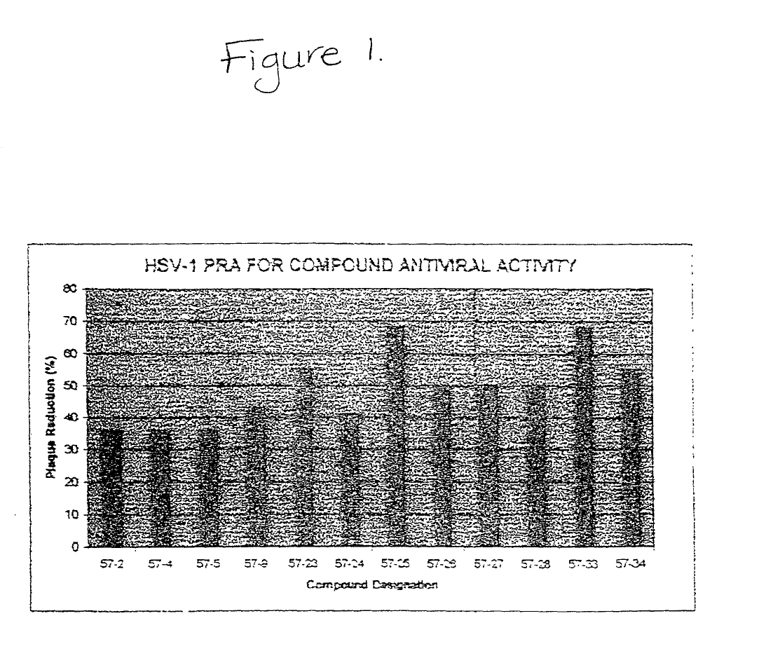

The in vitro antiviral activity of compounds of the invention against Herpes Simplex Virus Type 1 (HSV-1) were evaluated by a plaque reduction assay (PRA), ELISA and SDS PAGE/Western blotting. Vero cells (African green monkey kidney epithelial cells) were incubated with different MOI of virus and different concentrations of the antiviral compounds were used. Plaques were counted between 6 and 36 hours post-infection. In both ELISA and SDS/Western blotting, infected cells were disrupted at 6, 12, 24, 36, 48, 60 and 72 hours post-infection for HSV-1 specific antigen. Antigens corresponding to 2.5×10³/ml PFU were detectable 24 hours after infection while antigens derived from less than 1.5×10²/ml PFU were detectable 72 hours later. The test compounds showed different patterns of antiviral activities against HSV-1 the results of which are shown below in Tables 3-7 and FIGS. 1-5.

TABLE 3
HSV-1 plaque reduction assay and cytotoxicity assay results for the
specified compounds which correlate to the compounds shown in FIG. 1.

| Compound | % Plaque | Cytotoxicity |
|---|---|---|
| Number | Reduction | Concentration |
| 57-2 | 36 | >100 μM |
| 57-4 | 36 | >100 μM |
| 57-5 | 36 | >100 μM |
| 57-9 | 43 | ≧50 μM |
| 57-23 | 55 | ≧50 μM |
| 57-24 | 41 | ≦12.5 μM |
| 57-25 | 68 | ≧100 μM |
| 57-26 | 50 | ≧50 μM |
| 57-27 | 50 | ≧100 μM |
| 57-28 | 50 | ≧50 μM |
| 57-33 | 68 | >100 μM |
| 57-34 | 55 | ≦12.5 μM |

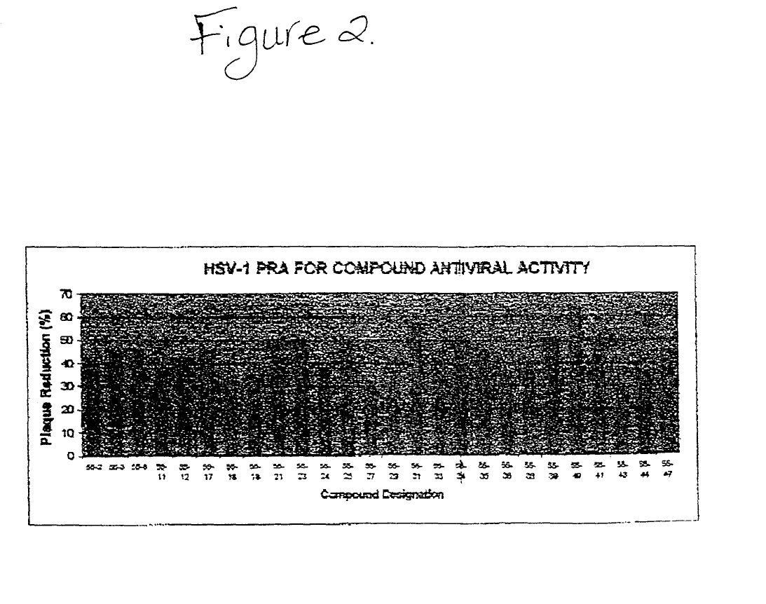

TABLE 4
HSV-1 plaque reduction assay and cytotoxicity assay results for the
specified compounds which correlate to the compounds shown in FIG. 2.

| Compound | % Plaque | Cytotoxicity |
|---|---|---|
| Number | Reduction | Concentration |
| 55-2 | 41 | >100 μM |
| 55-3 | 45 | >100 μM |
| 55-6 | 45 | >100 μM |
| 55-11 | 36 | >100 μM |
| 55-12 | 42 | >100 μM |
| 55-17 | 45 | >100 μM |
| 55-18 | 32 | >100 μM |
| 55-19 | 32 | >100 μM |
| 55-21 | 50 | >100 μM |
| 55-23 | 50 | >100 μM |
| 55-24 | 38 | >100 μM |
| 55-25 | 50 | >100 μM |
| 55-27 | 31 | >100 μM |
| 55-29 | 31 | >100 μM |
| 55-31 | 56 | >100 μM |
| 55-33 | 41 | >100 μM |
| 55-34 | 50 | ≦12.5 μM |
| 55-35 | 45 | ≧25 μM |
| 55-36 | 36 | ≧25 μM |
| 55-38 | 36 | ≦12.5 μM |
| 55-39 | 50 | ≦12.5 μM |
| 55-40 | 64 | ≦12.5 μM |
| 55-41 | 50 | ≧25 μM |
| 55-43 | 30 | ≧25 μM |
| 55-44 | 35 | ≧25 μM |
| 55-47 | 45 | |

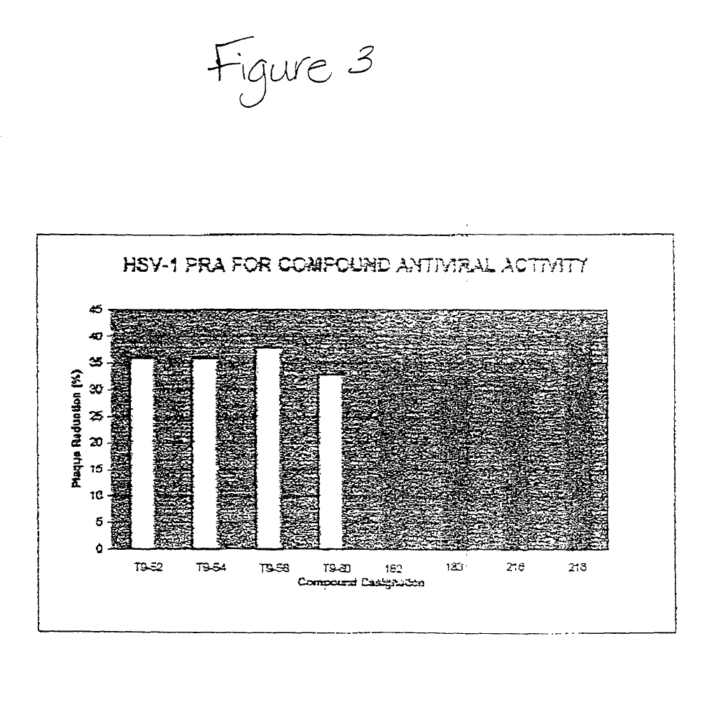

TABLE 5
HSV-1 plaque reduction assay and cytotoxicity assay results for the
specified compounds which correlate to the compounds shown in FIG. 3.

| Compound | % Plaque | Cytotoxicity |
|---|---|---|
| Number | Reduction | Concentration |
| T9-52 | 36 | >100 μM |
| T9-54 | 36 | >100 μM |
| T9-56 | 38 | >100 μM |
| T9-60 | 33 | >100 μM |
| 182 | 35 | >100 μM |
| 183 | 32 | >100 μM |
| 216 | 32 | >100 μM |
| 218 | 41 | >100 μM |

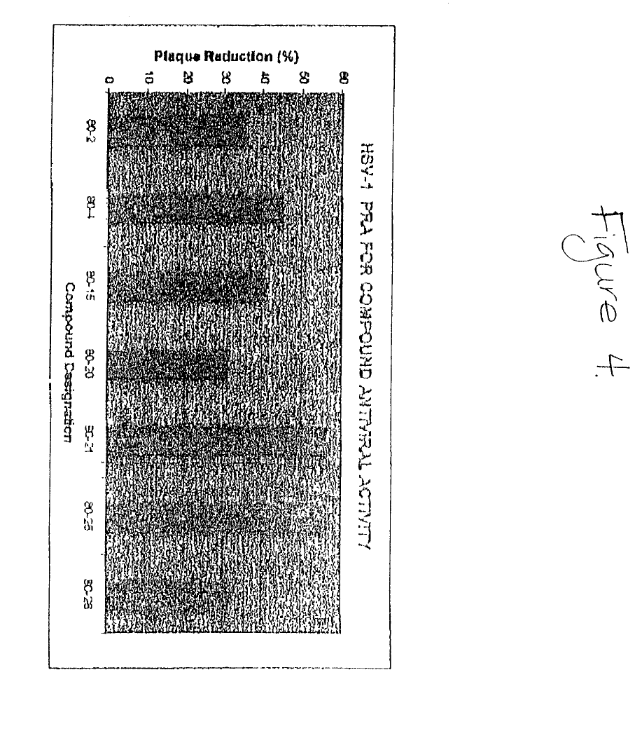

TABLE 6
HSV-1 plaque reduction assay and cytotoxicity assay results for the
specified compounds which correlate to the compounds shown in FIG. 4.

| Compound | % Plaque | Cytotoxicity |
|---|---|---|
| Number | Reduction | Concentration |
| 80-2 | 35 | >100 μM |
| 80-4 | 45 | >100 μM |
| 80-15 | 40 | >100 μM |
| 80-20 | 31 | >100 μM |
| 80-21 | 56 | >100 μM |
| 80-25 | 56 | >100 μM |
| 80-26 | 31 | >100 μM |

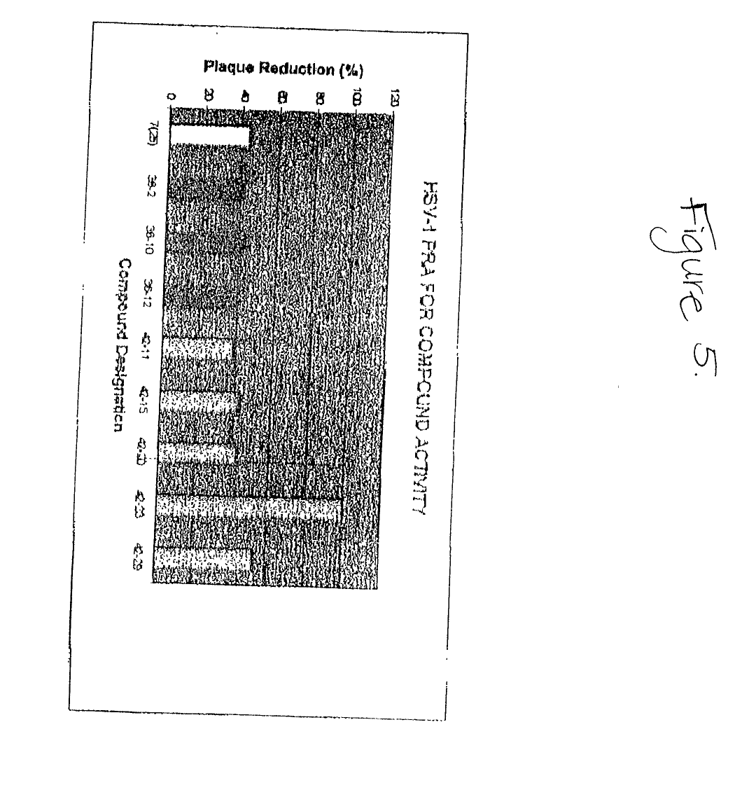

TABLE 7
HSV-1 plaque reduction assay and cytotoxicity assay results for the
specified compounds which correlate to the compounds shown in FIG. 5.

| Compound | % Plaque | Cytotoxicity |
|---|---|---|
| Number | Reduction | Concentration |
| 7(25) | 44 | ≧100 μM |
| 36-2 | 42 | ≧50 μM |
| 36-10 | 42 | ≧25 μM |
| 36-12 | 39 | ≧100 μM |
| 42-11 | 39 | ≧50 μM |
| 42-15 | 43 | ≧100 μM |
| 42-20 | 43 | ≧100 μM |
| 42-23 | 100 | ≦12.5 μM |
| 42-29 | 53 | ≦12.5 μM |

Compounds of the invention were also evaluated for antiviral activity against hepatitis-virus replication in cell-based assays. A number of active compounds were identified.

The invention has been described in detail with reference to preferred embodiments thereof. However, it will be appreciated that those skilled in the art, upon consideration of this disclosure, may make modifications and improvements within the spirit and scope of the invention as set forth in the following claims.

What is claimed is:

1. A compound library comprising two or more compounds of the following Formula I or I′: 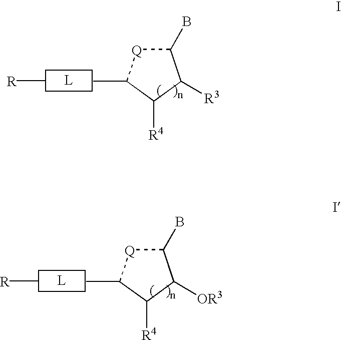
wherein L is a linking group such as e.g. an amide, ester, diester or the like, or an optionally substituted alkylene (e.g. C₁₋₂₀ alkylene), optionally substituted alkenylene (e.g., C₂₋₂₀ alkenylene) or alkynylene (e.g., C₂₋₂₀ alkynylene) having such groups either as a chain member of pendant to the chain, and which may be optionally substituted with one or more substituents selected from a group consisting of O, S, Se, NR¹NR²,CR¹CR², OR, SR and SeR, or an enzymatically reactive;
Q is carbon or a heteroatom such as O, S or N;
R is hydrogen or a hydroxyl group or an optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;
R¹, R², R³ and R⁴ are each independently selected from a group as defined by R;
B is optionally substituted adenine, optionally substituted thymidine, optionally substituted cytosine or an optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or a heterocyclic structure that is covalently linked to the sugar ring;
n=1 to 5;
and pharmaceutically acceptable salts thereof.

2. The library of claim 1 wherein at least one compound has a sugar group is in open chain form.

3. The library of claim 1 wherein an enantiomerically enriched mixture of a compound is present.

4. A compound library comprising two or more compounds of the following Formula II or II′: 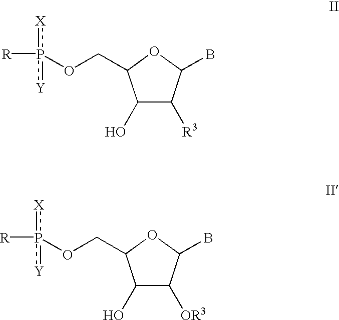
wherein X and Y are each independently selected from a group consisting of O, S, Se, NR¹NR², CR¹CR², OR, SR and SeR, or one or both of X and Y are an enzymatically reactive moiety;
R is hydrogen or an optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;
R¹, R² and R³ are each independently selected from a group as defined by R;
B is optionally substituted adenine, optionally substituted thymidine, optionally substituted cytosine or an optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or a heterocyclic structure that is covalently linked to the sugar ring;
and pharmaceutically acceptable salts thereof.

5. The library of claim 1 wherein at least one compound is of the following formula III or III′: 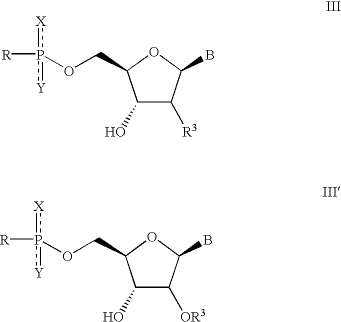
wherein X and Y are each independently selected from a group consisting of O, S, Se, NR¹NR², CR¹CR², OR, SR and SeR, or one or both of X and Y are an enzymatically reactive moiety;
R is hydrogen or an optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;
R¹, R² and R³ are each independently selected from a group as defined by R;
B is optionally substituted adenine, optionally substituted thymidine, optionally substituted cytosine or an optionally substituted guanine, preferably where the optional sub stituents are alkyl, carbocyclic aryl, or heteroaromatic or heteroalicyclic group preferably having from 1 to 3 separate or fused rings and 1 to 3 N, O or S atoms, or a heterocyclic structure that is covalently linked to the sugar ring;
and pharmaceutically acceptable salts thereof.

6. The library of any one of claims 1 through 6 wherein the library has been constructed using solution-phase synthesis.

7. A library of any one of claims 1 through 6 whereinn library is obtainable by a process comprising:
adding one or more reagents to a reaction vessel capable of agitation and containing a resin reaction support material;
agitating the reaction vessel during reaction of the reagents; and
centrifuging the reaction vessel and removing desired reaction materials therefrom.

8. The library of claim 7 wherein the library has been constructed using an automated solution-phase synthesis.

9. Use of the library of any one of claims 1 through 8 to find a specific interacting partner for a nucleic acid.

10. Use of the library of any one of claims 1 through 8 to find a specific interacting partner for a protein.

11. The use of claim 9 wherein the nucleic acid is RNA or DNA.

12. The use of claim 10 wherein the protein is an antibody, receptor or ligand.

13. A compound of the following Formula I or I′: 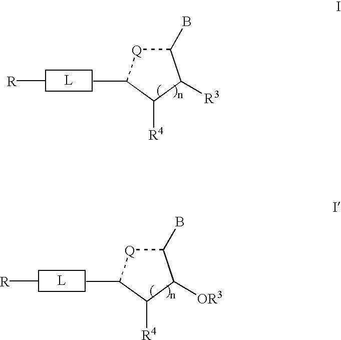
wherein L is a linking group such as an amide, ester, diester or the like which may be optionally substituted with one or more substituents selected from a group consisting of O, S, Se, NR¹NR², CR¹CR², OR, SR and SeR, or an enzymatically reactive moiety;
Q is carbon or a heteroatom such as O, S or N;
R is hydrogen or a hydroxyl group or an optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;
R¹, R², R³ and R⁴ are each independently selected from a group as defined by R;
B is optionally substituted adenine, optionally substituted thymidine, optionally substituted cytosine or an optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or or B is heteroaromatic or heteroalicyclic group other than an adenine, thymidine, cytosine or guanine;
N is an integer of from 1 (where to form a 5-membered ring as depicted or 5-membered acyclic group) to 5;
and pharmaceutically acceptable salts thereof.

14. A compound of claim 13 wherein the sugar group is in open chain form.

15. A compound of claim 13 wherein an enantiomerically enriched mixture of a compound is present.

16. A compound of the following Formula II or II′: 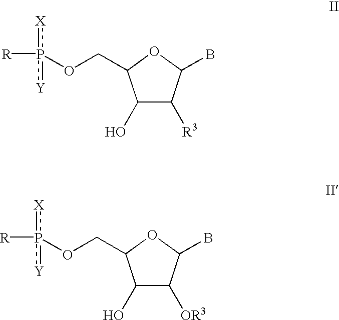
wherein X and Y are each independently selected from a group consisting of O, S, Se, NR¹NR², CR¹CR², OR, SR and SeR, or one or both of X and Y are an enzymatically reactive moiety;
R is hydrogen or an optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;
R¹, R² and R³ are each independently selected from a group as defined by R;
B is optionally substituted adenine, optionally substituted thymidine, optionally substituted cytosine or an optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or a heterocyclic structure that is covalently linked to the sugar ring;
and pharmaceutically acceptable salts thereof.

17. A compound of the following Formula III or III′: 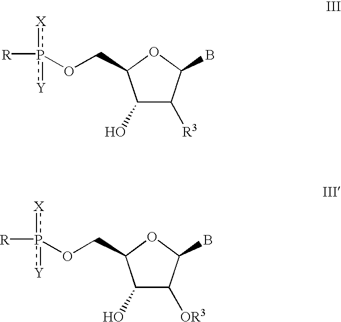
wherein X and Y are each independently selected from a group consisting of O, S, Se, NR¹NR², CR¹CR², OR, SR and SeR, or one or both of X and Y are an enzymatically reactive moiety;
R is hydrogen or an optionally substituted alkyl, optionally substituted alkenyl, optionally substituted alkynyl, optionally substituted aralkyl, optionally substituted cycloalkyl, optionally substituted cycloalkenyl, optionally substituted carbocyclic aryl, an optionally substituted mononucleotide, an optionally substituted polynucleotide, or an optionally substituted heteroaromatic or heteroalicyclic group preferably having from 1 to 3 separate or fused ring and 1 to 3 N, O or S atoms;
R¹, R² and R³ are each independently selected from a group as defined by R;
B is optionally substituted adenine, optionally substituted thymidine, optionally substituted cytosine or an optionally substituted guanine, preferably where the optional substituents are alkyl, carbocyclic aryl, or heteroaromatic or heteroalicyclic group preferably having from 1 to 3 separate or fused rings and 1 to 3 N, O or S atoms, or a heterocyclic structure that is covalently linked to the sugar ring;
and pharmaceutically acceptable salts thereof.

18. A method for treating virally infected cells comprising administering to the cells an anti-viral effective amount of a compound of any one of claims 13 through 17.

19. The method of claim 18 wherein the cells are infected with a herpes virus.

20. The method of claim 18 wherein the cells are infected with a cytomegalovirus.

21. A method for treating bacterially infected cells comprising administering to the cells an anti-bacteria effective amount of a compound of any one of claims 13 through 17.

22. A method for treating a mammal suffering from or susceptible to a viral infection, comprising administering to the mammal an anti-viral effective amount of a compound of any one of claims 13 through 17.

23. The method of claim 22 wherein the mammal is suffering from a herpes infection.

24. The method of claim 22 wherein the mammal is suffering from a cytomegalovirus infection.

25. A method for treating a mammal suffering from or susceptible to a bacterial infection, comprising administering to the mammal an anti-bacterial effective amount of a compound of any one of claims 13 through 17.

26. A pharmaceutical composition comprising a compound of any one of claims 13 through 17 and a pharmaceutically acceptable carrier.

27. A method for synthesis of a compound library, comprising:
adding one or more reagents to a reaction vessel capable of agitation and containing a resin reaction support material;
agitating the reaction vessel during reaction of the reagents; and
centrifuging the reaction vessel and removing desired reaction materials therefrom.

28. Use of the library or compound of any one of claims 1 through 8 or claims 13 through 17 to find a specific inhibitor for a viral kinase.

29. Use of the library or compound of any one of claims 1 through 8 or claims 13 through 17 to find a specific inhibitor for a viral polymerase.

30. Use of the library or compound of any one of claims 1 through 8 or claims 13 through 17 to find a specific compound which causes disruption of the association between a helicase-primase complex and a viral nucleic acid to which it is bound.

32. A method for treating a mammal suffering from or susceptible to a fungal infection, comprising administering to the mammal an anti-fungal effective amount of a compound of any one of claims 13 through 17.
